(19) United States
(12) Patent Application Publication
(10) Pub. No.: US 2023/0241088 A1
(43) Pub. Date: Aug. 3, 2023

(54) COMPOSITION FOR PREVENTING OR TREATING LUNG CANCER COMPRISING ORTHO-TOPOLIN RIBOSIDE AS ACTIVE INGREDIENT

(71) Applicant: CHUNG-ANG UNIVERSITY INDUSTRY-ACADEMY COOPERATION FOUNDATION, Seou (KR)

(72) Inventors: Hyung-Kyoon CHOI, Seoul (KR); Hwanhui LEE, Mungyeong-si (KR)

(73) Assignee: CHUNG-ANG UNIVERSITY INDUSTRY-ACADEMY COOPERATION FOUNDATION, Seou (KR)

(21) Appl. No.: 18/096,575

(22) Filed: Jan. 13, 2023

(30) Foreign Application Priority Data
Jan. 17, 2022 (KR) 10-2022-0006413

(51) Int. Cl. A61K 31/7076; A61P 35/00; A61P 11/00

(52) U.S. Cl. CPC A61K 31/7076; A61P 35/00; A61P 11/00

(57) ABSTRACT

The present disclosure relates to a composition for preventing or treating lung cancer, including ortho topolin riboside (oTR) as an active ingredient, wherein oTR showed the highest cytotoxicity among all tested compounds against NSCLC cells, oTR reduced synthesis of amino acid and pyrimidine as well as glycolytic function in NSCLC cells and xenograft tumors.

### CROSS-REFERENCE TO RELATED APPLICATION

This application claims the benefit of Korean Patent Application No. 10-2022-0006413 filed on Jan. 17, 2022, in the Korean Intellectual Property Office, the entire disclosure of which is incorporated herein by reference for all purposes.

### SEQUENCE LISTING

This application contains a Sequence Listing submitted via EFS-Web and hereby incorporated by reference in its entirety. The Sequence Listing is named SEQCRF_2280-434.xml, created on Jan. 10, 2023, and 4,096 bytes in size.

### BACKGROUND

### 1. Field of the Invention

The present disclosure relates to a composition for preventing or treating lung cancer, comprising ortho topolin riboside (oTR) as an active ingredient.

### 2. Description of the Related Art

Lung cancer has the highest incidence and mortality rates among all types of cancer worldwide. Over 75% of lung cancer cases have been diagnosed in patients at advanced stages (stage III or IV), which show poor survival rate. Lung cancer is generally classified as non-small cell lung cancer (NSCLC) and small cell lung cancer, and 80-85% of patients with lung cancer are diagnosed with NSCLC, which shows 5-years survival rate of only 5% at advanced stages. Overall, only 15-30% of patients with lung cancer have shown good response to chemotherapy using drugs, such as cisplatin, paclitaxel, gemcitabine, and etoposide. In addition, it has been reported that one of the treatment strategies, immunotherapy with targeting programmed cell death-1 or programmed death-ligand shows only 20-25% curability. Therefore, novel therapeutic agents and strategies are needed to reduce mortality rate and provide efficient treatment against NSCLC.

Approximately 25% of currently used anticancer drugs are derived from natural products, such as plants, and, in plants, plant hormones are involved in many processes such as growth, differentiation, bud formation, leaf expansion, and root development. Plant hormones, such as abscisic acid (ABA), auxins, and cytokinins, are produced in both plants and animals, and these have been known to be involved in cell proliferation and defensive responses. In particular, it has been reported that suppression of human tRNA isopentenyl transferase-1 (TRIT1) involved in cytokinin production showed lung tumorigenesis, and enhanced TRIT1 gene expression showed growth inhibition of mouse lung tumors.

Anticancer activities of cytokinins and their derivatives in myeloma, leukemia, melanoma, cervical carcinoma, and osteosarcoma cell lines have been reported previously. Among the cytokinins, ortho-topolin riboside (oTR) has been proposed as a potential anticancer agent with proven anti-proliferative activity against several cancer cell lines. oTR is a naturally occurring cytokinin in Populus×robusta leaves. In hepatoma cells, oTR-induced antiproliferative activity and apoptotic effects were observed through an increase in expression of cleaved caspase-3 and Bax known as pro-apoptotic indicators, and expression of a proliferative indicator such as phosphorylated ERK1/2 as well as anti-apoptotic indicators such as Bcl-2 and Bcl-xL. The antiproliferative activity of oTR was also shown in leukemia cells through inhibition of STAT3 signaling. However, oTR-induced metabolic alteration and anticancer effects on in vitro and in vivo models of NSCLC have not been reported yet. Alteration in intracellular metabolites and lipids has been known to be associated with many human cancers. Metabolomics and lipidomics-based research may provide insights into cancer-specific biomarkers and therapeutic targets.

### PRIOR ART DOCUMENT

### Patent Document

Chinese Patent Application Publication No. CN 107898804 A (published on Apr. 13, 2018)

### SUMMARY

### Problem to be Solved by the Invention

Accordingly, an object of the present disclosure is to provide a composition for preventing, ameliorating, or treating lung cancer, particularly, non-small cell lung cancer (NSCLC), comprising ortho topolin riboside (oTR) or a pharmaceutically acceptable salt thereof as an active ingredient.

### Means for Solving the Problem

The present disclosure provides a pharmaceutical composition for preventing or treating lung cancer, comprising ortho topolin riboside (oTR) or a pharmaceutically acceptable salt thereof as an active ingredient.

In addition, the present disclosure provides a health functional food composition for preventing or ameliorating lung cancer, comprising ortho topolin riboside (oTR) or a pharmaceutically acceptable salt thereof as an active ingredient.

### Effects of the Invention

The present disclosure relates to a composition for preventing or treating lung cancer, comprising ortho topolin riboside (oTR) as an active ingredient, wherein oTR showed the highest cytotoxicity among all tested compounds against NSCLC cells. oTR reduced synthesis of amino acid and pyrimidine as well as glycolytic function in NSCLC cells and xenograft tumors. Moreover, oTR reduced mitochondrial respiration function by inhibiting glutamine and fatty acid oxidation. Increased levels of phosphatidylcholine, phosphatidylethanolamine, and phosphatidylserine species suggested that oTR may act as a fatty acid oxidation inhibitor. In addition, the increased level of phosphatidylserine species implied that phosphatidylserine-mediated apoptosis occurred in oTR-treated NSCLC cells and xenograft tumor. The antiproliferative and apoptotic effects of oTR were mediated by the decreased level of p-ERK and p-AKT and the increased level of cleaved caspase-3, respectively. This is the first study to investigate the metabolic alterations and anticancer activity of oTR in in vitro and in vivo models of NSCLC. Accordingly, the present disclosure may be useful in the development of oTR-based therapeutics for NSCLC patients.

### BRIEF DESCRIPTION OF THE DRAWINGS

FIG. 1 shows a change in cell viability of NSCLC after 48 hours of oTR treatment. The bars and error bars on the graph represent the mean value and standard deviation of triplicate experiments, respectively. *, p<0.05; **, p<0.01; ***, p<0.001 (Student's t-test)

FIG. 2 shows results of tumor growth inhibition after oTR treatment in a xenograft mouse model. Changes in body weight (A), tumor growth (B), and images of tumor size (C) of the NSCLC xenograft model in control and oTR-treated (100 mg/kg and 150 mg/kg) groups are shown. The graphs are represented as mean±standard deviation (n=5). *, p<0.05 (Student's t-test for comparing control and 100 mg/kg oTR groups). #, p<0.05; ##, p<0.01 (Student's t-test for comparing control and 150 mg/kg oTR groups)

FIG. 3 shows volcano plots of altered metabolites and lipids in a cell system (A) and a xenograft model (B) induced by oTR treatment. Another model of volcano plots shows significant differences in metabolites and lipids between NSCLC and oTR-treated NSCLC. The gray dotted lines in the volcano plot represent the value of p (p<0.05). Cer, ceramide; PC, phosphatidylcholine; PE, phosphatidylethanolamine; PG, phosphatidylglycerol; PI, phosphatidylinositol; PS, phosphatidylserine; NS, no significant difference; AA, ascorbate; Ama, aminomalonate; Asp, aspartate; β-Ala, β-alanine; Cir, cirtrate; Cr, creatinine; Gluc, glucose; G6-P, glucose-6-phosphate; Glu, glutamate; Glyc, glycerol; Gly, glycine; HPX, hypoxanthine; Lac, lactate; Lys, lysine; Mal, malate; myo-ino, myo-inositol; Orn, ornithine; Phe, phenylalanine; Pro, proline; Putr, putrescine; pGlu, pyroglutamate; Ser, serine; Thr, threonine; Ura, uracil; and Uri, uridine.

FIG. 4 shows OPLS-DA-derived score plots and diagrams of permutation test in a cell system (A) and a xenograft model (B) of NSCLC and NSCLC after oTR treatment.

FIG. 5 shows results of alteration of glycolysis (A) and mitochondrial respiration (B) in NSCLC cells after oTR treatment. The NSCLC cells were treated with oTR (5, 10, and 50 μM) for 48 hours. Each graph represents mean values with error bars indicating standard deviation. (n=5, 5 biological replicates). Alteration of mitochondrial fuel capacity (C) in NSCLC cells treated with oTR (10 μM) (n=3, 3 biological replicates). *, p<0.05; **, p<0.01; ***, p<0.001 (Student's t-test). -, non-treated cells (control) were treated with an equal volume of DMSO; oTR, ortho topolin riboside; glycoPER, glycolytic proton efflux rate; OCR, oxygen consumption rate; and SRC, spare respiratory capacity

FIG. 6 shows the antiproliferative effect (A, D), apoptotic effect (B), and expression and quantification data of cell proliferation and apoptosis-related proteins (C) in NSCLC cells after oTR treatment. The cells were treated with oTR (5, 10, and 50 μM) for 48 hours in the presence or absence of siERK 1/2 (200 nM), siAKT (200 nM), U0126 (20 μM), and wortmannin (5 μM). Cells were transfected with siRNA for 24 hours or incubated with an inhibitor for 2 hours prior to oTR treatment. The effectiveness of the siRNA and inhibitors used was determined by Western blot assays of p-ERK and p-AKT (bottom panel). β-actin and GAPDH were used as loading controls. The graph shows fold-change values for the control group (non-treated cells). The error bars represent the standard error of the mean of 3 biological replicates. n=3; *, p<0.05; **, p<0.01; ***, p<0.001 (Student t-test). -, non-treated cells (as control) were treated with an equal volume of DMSO; oTR, ortho topolin riboside

FIG. 7 shows expression and quantification data (n=5) of cell proliferation and apoptosis-related proteins after oTR treatment in NSCLC tumor tissue. β-actin was used as a loading control. Graphs represent fold-change values relative to the control (non-treated xenograft tumor tissue). The error bars represent the standard error of the mean of 5 biological replicates. *, p<0.05; ***, p<0.001 (Student's t-test)

### DETAILED DESCRIPTION

The present disclosure provides a pharmaceutical composition for preventing or treating lung cancer, comprising ortho topolin riboside (oTR) or a pharmaceutically acceptable salt thereof as an active ingredient.

Preferably, the lung cancer may be a non-small cell lung cancer (NSCLC), but is not limited thereto.

Preferably, the composition may reduce synthesis of amino acid and pyrimidine and glycolytic functions.

Preferably, the composition may inhibit glutamine and fatty acid oxidation to reduce mitochondrial respiratory functions.

Preferably, the composition may induce phosphatidylserine (PS)-mediated apoptosis.

Preferably, the composition may decrease phosphorylated ERK1/2 (p-ERK) and phosphorylated AKT (p-AKT) and increase cleaved caspase-3.

The pharmaceutically acceptable salt may be selected from the group consisting of hydrochloride, bromate, sulfate, phosphate, nitrate, citrate, acetate, lactate, tartrate, maleate, gluconate, succinate, formate, trifluoroacetate, oxalate, fumarate, methanesulfonate, benzene sulfonate, p-toluenesulfonate, camphorsulfonate, sodium salt, potassium salt, lithium salt, calcium salt, and magnesium salt, but is not limited thereto.

The pharmaceutical composition or complex preparation of the present disclosure may be prepared using pharmaceutically suitable and physiologically acceptable adjuvants in addition to the active ingredient, wherein a solubilizer such as an excipient, disintegrant, sweetener, binder, coating agent, expander, lubricant, glydent, or flavoring agents may be used as the adjuvant. The pharmaceutical composition of the present disclosure may preferably be formulated as a pharmaceutical composition by including one or more types of pharmaceutically acceptable carriers in addition to the active ingredient for administration. The pharmaceutically acceptable carrier in the composition formulated into liquid solutions may be those that are sterile and suitable in vivo and used by mixing saline, sterile water, Ringer's solution, buffered saline, albumin injection solution, dextrose solution, maltodextrin solution, glycerol, ethanol, and one or more of these components, wherein other conventional additives such as an antioxidant, buffer, and bacteriostatic agent may be added as needed. In addition, by further adding a diluent, dispersant, surfactant, binder, and lubricant, it is possible to formulate into an injectable formulation such as an aqueous solution, suspension, and emulsion as well as a pill, capsule, granule, or tablet.

The pharmaceutical preparation form of the pharmaceutical composition of the present disclosure may be a granule, acid, coated tablet, tablet, capsule, suppositories, syrup, juice, suspension, emulsion, dropper or injectable liquid, and sustained release preparation of active compounds. The pharmaceutical composition of the present disclosure may be administered in a conventional manner through intravenous, intraarterial, intraperitoneal, intramuscular, intrasteral, transdermal, intranasal, inhaled, topical, rectal, oral, intraocular, or intradermal routes. An effective amount of the active ingredient of the pharmaceutical composition of the present disclosure refers to an amount required for prevention or treatment of a disease. Thus, the amount may be adjusted by various factors such as the type of a disease, the severity of a disease, the type and content of the active ingredient and other ingredients included in the composition, the type of formulation and age, weight, general health status, sex, and diet of a patient, administration time, administration route and secretion rate of the composition, treatment period, and concomitant drug.

In addition, the present disclosure provides a health functional food composition for preventing or ameliorating lung cancer, comprising ortho topolin riboside (oTR) or a pharmaceutically acceptable salt thereof as an active ingredient.

The health functional food composition of the present disclosure may be provided in the form of powder, granules, tablets, capsules, syrups, or beverages, wherein the health functional food composition may be used in combination with other foods or food additives in addition to the active ingredient and appropriately used according to conventional methods. The mixing amount of the active ingredient may be suitably determined according to the purpose of use thereof, for example, prevention, health, or therapeutic treatment. The effective dose of the active ingredient included in the health functional food composition may be applied according to the effective dose of the pharmaceutical composition, but it may be less than the range in the case of long-term intake for health and hygiene purposes or for health control, wherein it is clear that the active ingredient may be used in an amount greater than the range since there is no problem in terms of safety.

There are no special restrictions in the types of health foods, and examples may include meat, sausage, bread, chocolate, candy, snacks, confectionery, pizza, ramen, other noodles, chewing gum, dairy products including ice cream, various soups, beverages, tea, drinks, alcoholic beverages, and vitamin complexes.

Hereinafter, to help the understanding of the present disclosure, example embodiments will be described in detail. However, the following example embodiments are merely illustrative of the content of the present disclosure, and the scope of the present disclosure is not limited to the following example embodiments. The example embodiments of the present disclosure are provided to more completely explain the present disclosure to those of ordinary skill in the art.

### Experimental Example

The following experimental examples are intended to provide experimental examples commonly applied to each example embodiment according to the present disclosure.

1. Chemicals and Reagents

High-performance liquid chromatography grade methanol, ethanol, and water were purchased from Thermo Fisher Scientific (Pittsburgh, Pa., USA). Butylated hydroxytoluene (BHT), dimethyl sulfoxide (DMSO), potassium iodide (KI), wortmannin, and trichloroacetic acid (TCAA) were purchased from Sigma-Aldrich (St. Louis, Mo., USA). U0126 was purchased from Calbiochem (San Diego, Calif., USA). 3-(4,5-dimethylthiazol-2-yl)-2,5-diphenyl tetrazolium bromide (MTT) was purchased from Alfa Aesar (Ward Hill, Mass., USA).

2. Plant Hormones

Kinetin riboside (KR), N⁶-benzyladenosine (N⁶b), oTR, SS-homocastasterone (SS-HCS), and Gerald Rosebery-24 (GR-24) were purchased from OlChemIm Ltd. (Olomouc, Czech Republic). N⁶-isopentenyladenosine (N⁶i) was purchased from Toronto Research Chemicals Inc. (Toronto, ON, Canada). ABA, coronatine (Cor), ethephon, indole acetic acid (IAA), and methyl jasmonate (MJ) were purchased from Sigma-Aldrich. Cor, IAA, KR, N⁶b, N⁶i, oTR, SS-HCS, and GR-24 were dissolved in DMSO, and ABA, ethephon, and MJ were dissolved in methanol, water, and ethanol, respectively.

3. Cell Cultures and Cell Viability Analysis

Human NSCLC cell lines A549, H1437, H2087, H522, and HCC827 were obtained from the Korean Cell Line Bank (KCLB, Seoul, Korea). These cells were cultured in RPMI-1640 medium (Gibco, N.Y., USA) supplemented with 10% FBS (Gibco) and 1% penicillin-streptomycin (HyClone Laboratories, Logan, Utah, USA). All cell lines were cultured under standard conditions (5% CO₂, humid environment, 37° C.). A549 cells were seeded at a density of 1×10⁴ cells/well and H1437, H2087, H522, HCC827, and MRCS cells at a density of 2×10⁴ cells/well in 96-well plates and were cultured until cells reach approximately 70-80% confluency. The NSCLC cells were treated with plant hormones for 48 hours. Stock solution of each plant hormone was diluted with culture medium, wherein concentrations of DMSO, water, methanol, and ethanol in the culture medium were below 1% (v/v). The half-maximal inhibitory concentration (IC₅₀) was calculated using CompuSyn software (ComboSyn Inc., Paramus, N.J., USA).

4. Measurement of the Effect of oTR on Tumor Growth in a Xenograft Mouse Model

To evaluate the anticancer effect of oTR in tumor tissues, a tumor xenograft model was established by injecting 2.5×10⁶ A549 cells into the flanks of nude mice (female, BALB/cSlc-nu/nu). After the tumor volume reached above 100 mm³ after approximately 3 weeks, the mice were randomized into negative controls and experimental groups (5 mice were used per group). Mice were then injected with oTR solution into the tumor area twice a week for 3 weeks. oTR was dissolved in saline:DMSO (1:1, v/v), and 50 μL of oTR solution was injected into mice in a dose of 100 mg/kg or 150 mg/kg. These doses indicated no acute toxicity in the previous study. Negative controls were injected with saline:DMSO (1:1, v/v) without oTR. Tumor volume, mouse body weight, and mouse survival were recorded twice a week before injecting oTR. Tumor length (a) and width (b) were measured using calipers and tumor volume (mm³) was calculated by =(a×b²)/2.

5. Sample Collection and Extraction for Mass Spectrometry (MS) Analysis

The cells were seeded at a density of 2×10⁵ cells/mL in a 6-well plate and treated with oTR (10 μM) for 48 hours. The cells were pooled from 3 wells in the 6-well plate and centrifuged at 1,000×g at 4° C. for 2 minutes. The cells were frozen in liquid nitrogen and stored at −70° C. until analysis. The cells were lysed by two freeze-thaw cycles using liquid nitrogen and 37° C. water bath. Protein concentrations were determined using the Bio-Rad protein assay kit (Thermo Scientific, Rockford, Ill.). Bovine serum albumin (BSA) was used as a standard for normalization. The cells were freeze-dried and stored at −70° C. until additional analysis.

The tumor tissue was weighed above 25 mg, and 0.3 mL of ice-cold methanol containing 0.1% BHT was added, followed by homogenization for 1 minutes. Homogenized samples were centrifuged at 10,000×g for 15 minutes at 4° C. The supernatant corresponding to 10 mg of the tissue was used, and metabolites and lipids were extracted using modified Folch method.

6. Gas Chromatography-Mass Spectrometry (GC-MS) and Nano Electrospray Ionization-Mass Spectrometry (nanoESI-MS) Analyses

To perform metabolite and lipid analyses of the extracted samples of NSCLC cells and xenograft tumor tissues, GC-MS and nanoESI-MS analyses were conducted according to a previously described method. For quality control (QC), the equal volume of mixture was taken from each sample to be analyzed.

7. Glycolytic and Mitochondrial Function Analyses

Mitochondrial and glycolytic functions were assessed using Seahorse XFe24 analyzer (Agilent Technologies, Lexington, Mass., USA). A549 cells were seeded in a cell culture plate at a density of 1.5×10⁴ cells/well. Cells were treated with oTR (5, 10, and 50 μM) for 48 hours after achieving approximately 70-80% confluency. The glycolytic and mitochondrial functions were determined by measuring the proton efflux rate (PER) and oxygen consumption rate (OCR), respectively. Glycolytic PER (glycoPER) was obtained by subtracting mitochondria-derived protons from the total PER. The analyses were conducted according to the manufacturer's instructions.

8. Measurement of H₂O₂ Content

A549 cells were seeded at a density of 2×10⁵ cells/mL in a 6-well plate and treated with oTR (5, 10, and 50 μM) or an equal volume of DMSO (control) for 48 hours. H₂O₂ content was measured according to a previously described method with some modifications added. Briefly, the cell pellets were extracted with 1 mL of 0.1% TCAA, and the extracts were homogenized and centrifuged at 15,000×g at 4° C. for 10 minutes. Potassium phosphate buffer (10 mM; 0.5 mL) and 1 M KI (1 mL) were added to 0.5 mL of the supernatant. The mixture was then incubated for 15 minutes at room temperature in the dark and measured at 390 nm. For normalization, protein concentration was determined using Bio-Rad protein assay kit with BSA as a standard.

9. RNA Interference

The siRNA sequences for ERK1/2 (5′-CCCUGACCCGUCUAAUA-3′ SEQ ID NO: 1), AKT (5′-GAAGGAAGU CAUCGUGCCCAA-3′ SEQ ID NO: 2), and scrambled siRNA (SN-1001-CFG) were purchased from Bioneer (Daejeon, Korea). Cells were transfected with siRNA using Lipofectamine 3000 (Invitrogen, Carlsbad, Calif., USA) according to the manufacturer's instructions.

10. Cell Proliferation Assay and Apoptosis Analysis

The 5-bromo-2-deoxyuridine (BrdU) cell proliferation assay was performed according to a previously described method. A549 cells (1×10⁴) were grown to approximately 70-80% confluency in 96-well plates and treated with oTR (5, 10, and 50 μM) for 48 hours in the presence or absence of U0126, wortmannin, siERK1/2, and siAKT. Cell proliferation was measured using the Cell Proliferation ELISA, BrdU (colorimetric) kit (Roche Diagnostics, Penzberg, Germany), according to the manufacturer's instructions.

To detect apoptotic cells, flow cytometric analysis was performed using Annexin V-FITC/propidium iodide double staining of NSCLC cells. A549 cells (4×10⁵) were grown to approximately 70-80% confluency in 60 mm dish and treated with oTR (5, 10, and 50 μM) for 48 hours. The cells were stained using the FITC Annexin V Apoptosis Detection Kit (556547, BD Biosciences, Franklin Lakes, N.J., USA) as previously described.

11. Immunoblot Analysis

The immunoblot analysis was carried out according to a previously described method. A549 cells (4×10⁵) were grown to approximately 70-80% confluency and treated with oTR (5, 10, and 50 μM) for 48 hours. The cell lysates and tissue lysates were prepared and subjected to SDS-PAGE for immunoblotting with the respective antibodies. Anti-caspase-3 (9662), anti-p-AKT (4058), and anti-AKT (9272) antibodies were purchased from Cell Signaling Technology (Danvers, Mass., USA). Anti-ERK1/ERK2 (MAB1576) and anti-p-ERK1/ERK2 (AF1018) were purchased from R&D Systems (Minneapolis, Minn., USA). Anti-LC-III (NB100-2331) antibody was purchased from Novus Biologicals (Littleton, Colo., USA). Anti-β-actin (sc-47778) and anti-GAPDH (sc-47724) antibody was purchased from Santa Cruz Biotechnology (Santa Cruz, Calif., USA).

12. Data Processing and Statistical Analyses

Raw data files of the lipid analysis (*.raw) were converted into *.mzXML format using the Proteo Wizard MSConvert software. The metabolite and lipid mass spectra were converted using the Expressionist® MSX software (version 2013.0.39, Genedata, Basel, Switzerland) for data processing. The data obtained from NSCLC cells were divided by the peak intensity of the internal standard and total protein content for normalization. The data obtained from the NSCLC xenograft model were divided by the peak intensity of the internal standard for normalization. The data obtained thereby were used for fold-change analysis using the MetaboAnalyst (version 5.0; http://www.metaboanalyst.ca). SPSS software (version 26, IBM, Somers, N.Y.) was used for significance examination for the results, and identification was conducted by Student's t-test and Mann-Whitney test. All data were mean-centered and pretreated to Pareto scaling for principal component analysis (PCA) and orthogonal partial least-squares discriminant analysis (OPLS-DA), performed using the SIMCA-P software (version 15.0.2, Sartorius Stedim Data Analytics AB, Umea, Sweden). Cross validations and permutation tests (100 times) were performed to evaluate the goodness of fit value and predictability of the OPLS-DA model. Volcano plots were generated using the GraphPad Prism 9.0.

### <Example 1> Inhibition of Cell Viability of Human NSCLC Cells by oTR

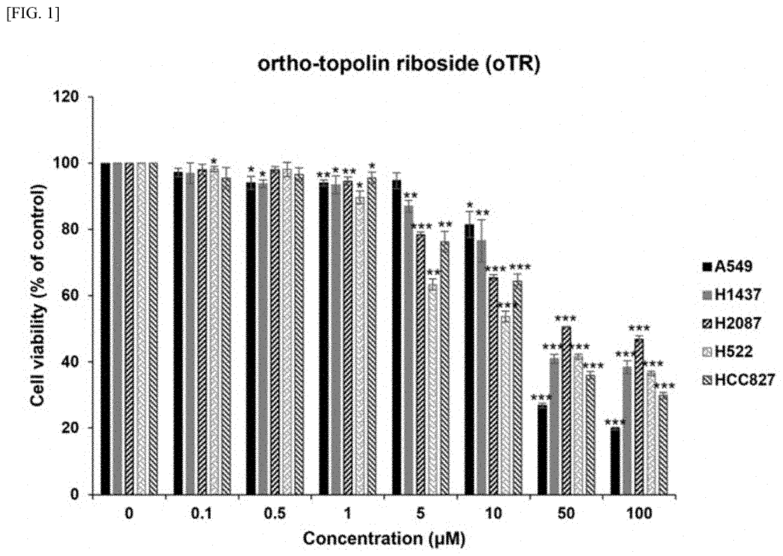

The inhibitory effect of plant hormones and their derivatives was investigated on 5 NSCLC cell lines (A549, H1437, H2087, H522, and HCC827). The NSCLC cells were treated with ABA, Cor, ethephon, GR-24, IAA, KR, MJ, N6b, N6i, oTR, and SS-HCS at concentrations of 0.1, 0.5, 1, 5, 10, 50, and 100 μM. Cell viability and IC50 values for 48 hours were investigated by performing the MTT assay. Among the tested plant hormones and derivatives, cytokinins (KR, N6b, N6i, oTR) showed IC50 values less than 100 μM in NSCLC cells compared to other plant hormones and derivatives (Table 1). In particular, oTR showed the lowest IC50 values among all tested plant hormones and derivatives (Table 1). The IC50 values of oTR were 34.33±4.40, 49.97±6.55, 51.37±5.74, 26.13±2.47, and 29.70±2.56 μM for A549, H1437, H2087, H2087, and H522, respectively. As shown in FIG. 1, oTR treatment reduced the viability of NSCLC cells.

TABLE 1

| Phytohormone | NSCLC cells | | | | |
|---|---|---|---|---|---|
| and derivatives | A549 | H1437 | H2087 | H522 | HCC827 |
| ABA | >100 | >100 | >100 | >100 | >100 |
| Cor | >100 | >100 | >100 | >100 | >100 |
| Ethephon | >100 | >100 | >100 | >100 | >100 |
| GR-24 | >100 | >100 | >100 | >100 | >100 |
| IAA | >100 | >100 | >100 | >100 | >100 |
| KR | 12.26 ± 1.42 | 76.79 ± 13.74 | 98.51 ± 6.72 | 72.98 ± 10.86 | 33.13 ± 0.14 |
| MJ | >100 | >100 | >100 | >100 | >100 |
| N⁶b | 28.15 ± 0.63 | 70.27 ± 17.82 | >100 | >100 | 48.96 ± 21.39 |
| N⁶i | 26.69 ± 2.58 | 90.42 ± 10.72 | 95.98 ± 7.45 | >100 | 39.85 ± 3.19 |
| oTR | 34.33 ± 4.40 | 49.97 ± 6.55 | 51.37 ± 5.74 | 26.13 ± 2.47 | 29.70 ± 2.56 |
| SS-HCS | 87.55 ± 15.45 | 34.72 ± 2.04 | 85.38 ± 8.81 | >100 | 72.28 ± 13.61 |

### <Example 2> Inhibition of Tumor Growth in the Xenograft Mouse Model by oTR

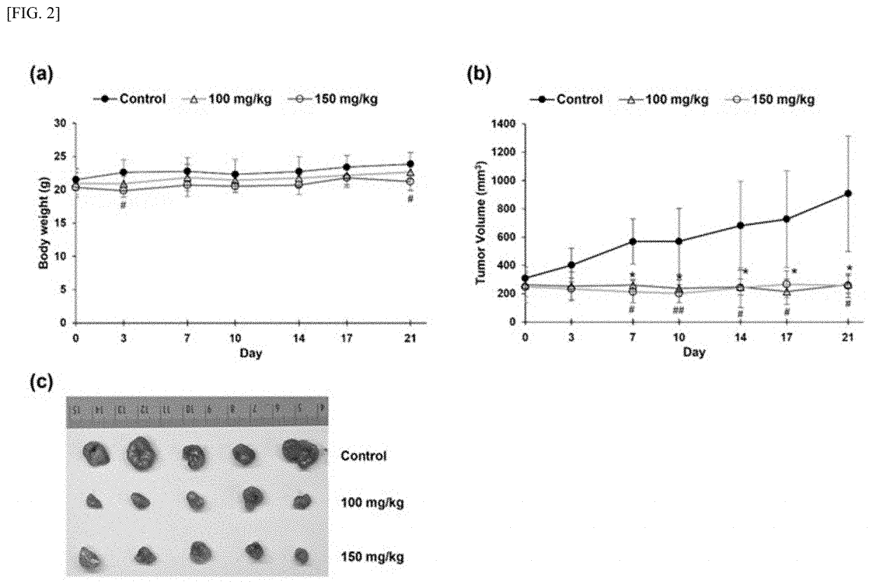

Tumor xenograft mice were treated with oTR at two doses (100 mg/kg and 150 mg/kg). The tumor volume was measured twice a week for 3 weeks to evaluate the therapeutic effects of the drug in vivo. The body weight of the mice in the control and oTR-treated groups was measured twice a week before injecting oTR (FIG. 2A). Compared to that of the control group, the body weight of the oTR-treated group (100 mg/kg) did not significantly change, whereas the oTR-treated group (150 mg/kg) showed a significant decrease in body weight on day 3 and day 21. The body weight did not reduce by more than 5% of the initial body weight (data not shown). These data demonstrated that oTR treatment and tumor size did not significantly affect weight loss. The tumor volume was significantly decreased after the second treatment (day 7) in the oTR-treated groups (100 mg/kg and 150 mg/kg) compared to that in the control group (FIGS. 2B and C). However, the oTR-induced changes in the tumor volume did not decrease in a dose-dependent manner.

### <Example 3> Altered Metabolic and Lipidomic Profiles in Human NSCLC Cells and Xenograft Tumor Tissues Induced by oTR

oTR-induced alterations of metabolic and lipidomic profiles were analyzed using 5 NSCLC cell lines (A549, H1437, H2087, H522, and HCC827) and xenograft tumor tissues by performing GC-MS and nanoESI-MS analyses. The relative changes in identified metabolites and intact lipid species are shown as fold-changes. Myo-inositol, amino acids (β-alanine, aspartate, serine, threonine, glutamate, glycine, proline, and tyrosine), organic acids (malate and lactate), pyrimidines (uracil and uridine), creatinine, hypoxanthine, and putrescine decreased in oTR-treated NSCLC cells compared to those in NSCLC cells. These metabolites were the outcomes of consistent and significant alterations in 3 NSCLC cell lines compared to those of respective controls. In particular, the lactate level significantly decreased in all NSCLC cell lines after oTR treatment.

In xenograft tumor tissues, greater changes in metabolite levels were observed in the high-dose group (150 mg/kg oTR) than those in the low dose group (100 mg/kg oTR) compared with the control group. oTR-induced significant reduction in the level of aspartate, ascorbate, glucose, glutamate, ornithine, phenylalanine, proline, serine, and threonine was observed in the oTR (150 mg/kg) group compared with those in the control group. Citrate, ornithine, and uridine significantly decreased in both oTR groups. However, β-Alanine, aminomalonate, glycerol, glycine, and uracil increased in both oTR groups.

In the case of lipid, phosphatidylcholine (PC) and plasmenyl-phosphatidylethanolamine (plasmenyl-PE) were detected in the positive ion mode, whereas ceramide (Cer), PE, plasmenyl-PE, phosphatidylglycerol (PG), phosphatidylinositol (PI), and phosphatidylserine (PS) were detected in the negative ion mode. The PC, PE, and PS species and Cer 18:1/22:0 increased in oTR-treated NSCLC cells, whereas the levels of PG and PI species decreased in oTR-treated NSCLC cells.

Moreover, an increase in PC, PE, PG, and PI species was observed in the xenograft tumor tissues treated with 100 mg/kg and 150 mg/kg of oTR. The PS species increased only in the 150 mg oTR-treated group compared with the control.

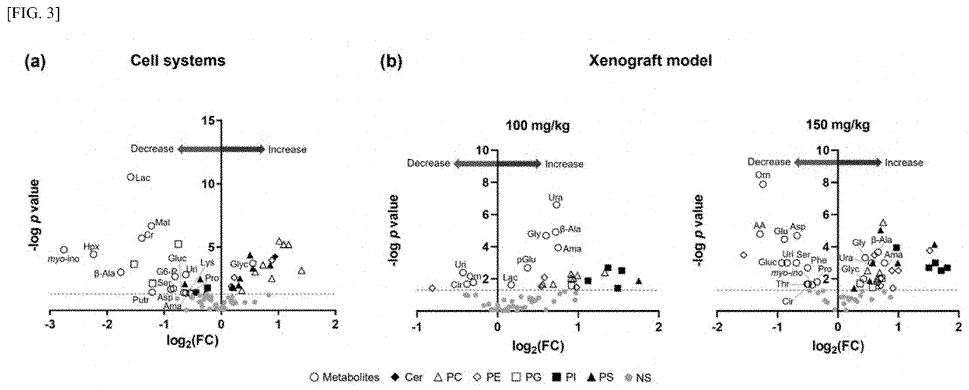

The volcano plot indicated by different dots showed significant changes in metabolites and lipids based on the fold-change and p values (<0.05) of oTR-treated NSCLC cells and the xenograft tumor tissue compared with those of the respective controls (FIG. 3). Overall, a similar pattern of metabolite and lipid profiles was observed in the cell systems and 150 mg/kg oTR-treated group. Aspartate, glucose, myo-inositol, proline, serine, and uridine significantly decreased in the cell and 150 mg/kg oTR-treated group. In particular, uridine was observed to be significantly decreased in the cell and oTR-treated xenograft models (100 and 150 mg/kg). PC, PS, and PE species significantly increased in the cell and xenograft models following oTR treatment. However, the levels of β-alanine, aminomalonate, lactate, uracil, PG, and PI species were differently expressed in the cell and xenograft models after oTR treatment.

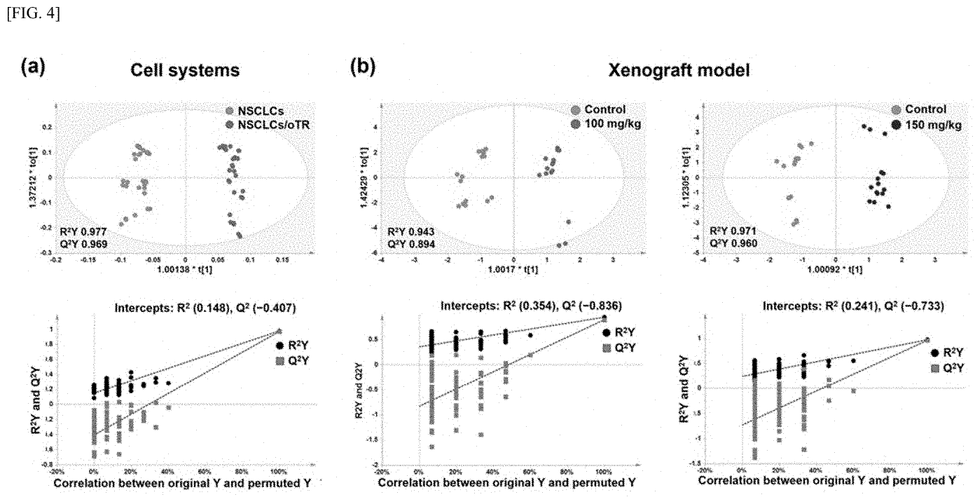

oTR-induced alterations in metabolite and lipid profiles in the cells and xenograft models were shown by PCA and OPLS-DA. QC samples were clustered in the PCA-derived score plot, indicating that the analytical methods showed high reproducibility and stability in both cell and xenograft models. OPLS-DA was employed to maximize intergroup separation based on metabolite and intact lipid species profiles (FIG. 4). As shown in FIGS. 4A and 4B, NSCLC (cells and tumor tissues) and oTR-treated NSCLC (cells and tumor tissues) were clearly distinguished. R²Y (0.977, cells; 0.943, 100 mg/kg oTR; 0.971, 150 mg/kg oTR) and Q²Y (0.969, cells; 0.894, 100 mg/kg oTR; 0.960, 150 mg/kg oTR) indicate high reliability and predictability of the model. Permutation tests were performed to assess the validity of the model, and the R²Y intercept and Q²Y intercept values obtained in the present disclosure were (0.148, cells; 0.354, 100 mg/kg oTR, 0.241; 150 mg/kg oTR) and (−0.407, cells; −0.836, 100 mg/kg oTR; −0.733, 150 mg/kg oTR). The values shown above satisfied the criteria of R²Y intercept <0.3-0.4 and Q²Y intercept <0.05. The variable importance in projection (VIP) score of the OPLS-DA model indicate the importance of each variable for sample separation, and variables with VIP score >0.7 are considered as sufficiently influential for the model. Amino acid, lactic acid, glucose, and lipids (PC, PS, PI, and PG species) were indicated as influential compounds for group separation.

### <Example 4> oTR-Induced Inhibition of Glycolytic and Mitochondrial Respiration Functions in Human NSCLC Cells

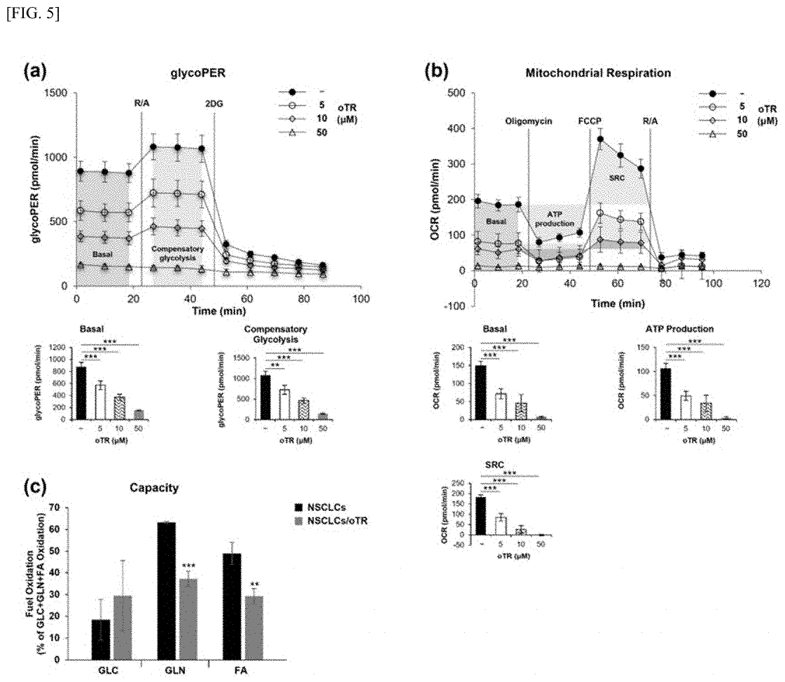

To investigate real-time changes in cellular metabolic functions, glycoPER (pmol H+/min) and OCR were analyzed (FIG. 5). GlycoPER reflects glycolytic functions, and OCR reflects mitochondrial respiration. As shown in FIG. 5, the glycolytic and mitochondrial respiration functions were inhibited in A549 cells after oTR treatment in a dose-dependent manner (5, 10, and 50 μM). A significant decrease was observed in basal and compensatory glycolytic rates in oTR-treated NSCLC cells (FIG. 5A). Measurement of compensatory glycolysis was achieved by blocking oxidative phosphorylation using rotenone/antimycin.

As shown in FIG. 5B, OCR was reduced in NSCLC cells after oTR treatment. To measure ATP production, oligomycin, an ATPase inhibitor, was added to the cells to inhibit mitochondria-derived ATP synthesis. Then, when carbonyl cyanide-4-(trifluoromethoxy)phenylhydrazone (FCCP), a mitochondrial oxidative phosphorylation uncoupler, was added to the cells, the OCR reached the maximal level, indicating maximal mitochondrial respiration. Spare respiratory capacity (SRC) indicates the amount of extra ATP produced by oxidative phosphorylation when energy demand of the cells increases suddenly. Significant reduction in basal OCR, ATP production, and SRC was observed in A549 cells after oTR treatment compared with that in the untreated A549 cells. To determine the mitochondrial fuel source (glucose, glutamine, and fatty acid) that was better utilized in NSCLC cells, investigation was performed on the OCR of each fuel source in the absence and presence of each inhibitor. UK5099 (for glucose), BPTES (for glutamine), and etomoxir (for fatty acid). As shown in FIG. 5C, glutamine and fatty acid oxidations were significantly reduced, but glucose oxidation did not differ between NSCLC cells and oTR-treated NSCLC cells. That indicates that oTR inhibited mitochondrial respiration by reducing glutamine and fatty acid oxidation in NSCLC cells.

The H₂O₂ content in A549 cells after oTR treatment (5, 10, and 50 μM) was identified. oTR-treated A549 cells showed significantly increased H₂O₂ content compared to the untreated A549 cells in a dose-dependent manner.

### <Example 5> oTR-Induced Antiproliferative Activity and Apoptotic Effects

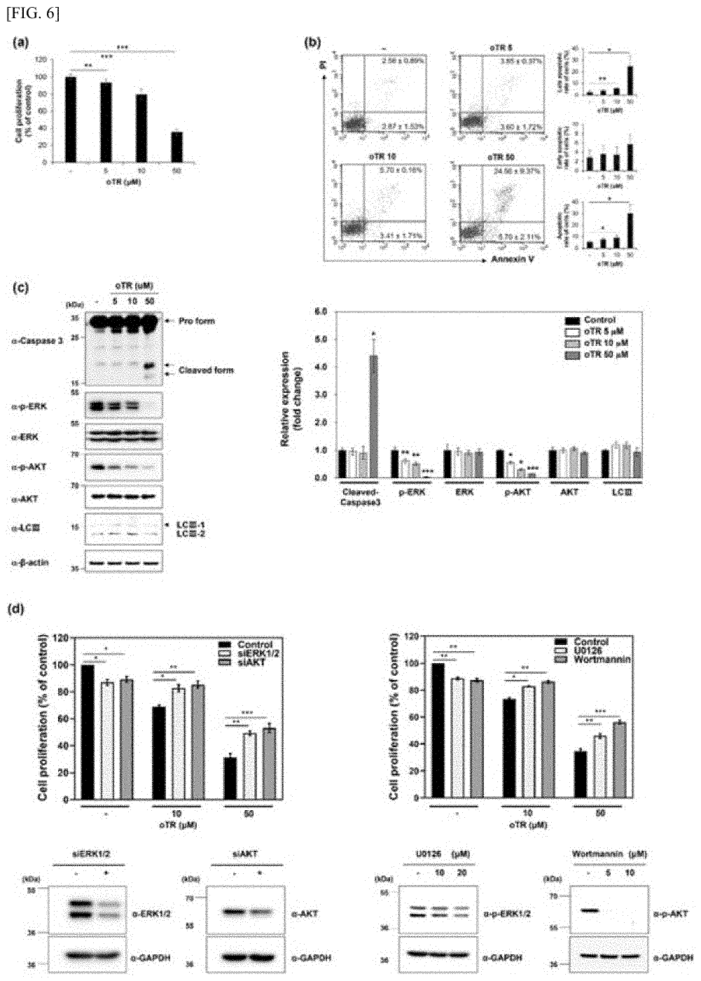

BrdU cell proliferation assay was performed to investigate the antiproliferative effect of oTR on NSCLC cells. It was observed that oTR significantly inhibited cell proliferation in a dose-dependent manner (FIG. 6A), which is consistent with the results of the cell viability assay (FIG. 1). In addition, the percentage of late apoptotic cells increased by 5.70±0.16% and 24.56±9.37% compared to the untreated NSCLC cells after treatment with 10 and 50 μM oTR respectively (FIG. 6B). The ERK and AKT pathways are known to be associated with tumor growth and cell proliferation. oTR treatment significantly decreased phosphorylated ERK1/2 (p-ERK) and phosphorylated AKT (p-AKT) in NSCLC cells. Cleaved caspase-3, an apoptotic marker, significantly increased in NSCLC cells treated with 50 μM oTR (FIG. 6). However, the level of LC-III, an autophagy marker, did not change after oTR treatment (FIG. 6C). To further determine whether activation of ERK and AKT is an important signaling mediator for oTR-induced cell proliferation inhibition, ERK or AKT was silenced or inhibited using siRNA or inhibitors, respectively. oTR-induced antiproliferative activity was significantly inhibited in ERK or AKT knockdown NSCLC cells (FIG. 6D). Consistently, treatment of cells with ERK inhibitors (U0126) or AKT inhibitors (wortmannin) significantly weakened the antiproliferative effect of oTR (FIG. 6D).

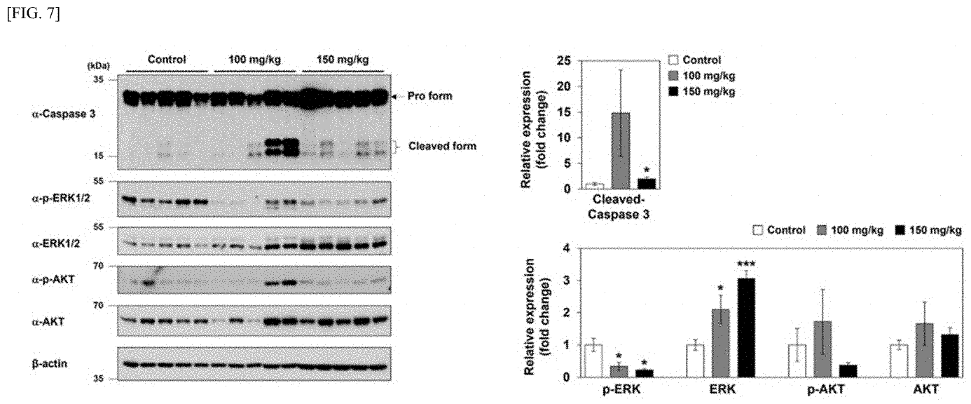

As shown in FIG. 7, a significant decrease in p-ERK expression and significant increase in ERK expression were observed in oTR-treated NSCLC tumor tissues. However, no significant changes were observed in p-AKT and AKT expressions after oTR treatment. In addition, significantly increased expression of cleaved caspase-3 was observed in the oTR (150 mg/kg)-treated group (FIG. 7).

As described above, since a specific part of the content of the present disclosure is described in detail, for those of ordinary skill in the art, it is clear that the specific description is only a preferred embodiment, and the scope of the present disclosure is not limited thereby. Thus, the substantial scope of the present disclosure will be defined by the appended claims and their equivalents.

What is claimed is:

1. A method of preventing or treating lung cancer, comprising:
administering a pharmaceutical composition comprising ortho topolin riboside (oTR) or a pharmaceutically acceptable salt thereof as an active ingredient to a subject.

2. The method of claim 1, wherein the lung cancer is non-small cell lung cancer (NSCLC).

3. The method of claim 1, wherein the composition reduces synthesis of amino acid and pyrimidine as well as glycolytic function.

4. The method of claim 1, wherein the composition reduces mitochondrial respiration function by inhibiting glutamine and fatty acid oxidation.

5. The method of claim 1, wherein the composition induces phosphatidylserine (PS)-mediated apoptosis.

6. The method of claim 1, wherein the composition decreases phosphorylated ERK1/2 (p-ERK) and phosphorylated AKT (p-AKT) and increases cleaved caspase-3.

7. A method of preventing or ameliorating lung cancer, comprising:
administering a health functional food composition comprising ortho topolin riboside (oTR) or a pharmaceutically acceptable salt thereof as an active ingredient to a subject.


SEQUENCE LISTING (2 sequences)

SEQ ID NO: 1 - RNA, 17 nt, synthetic construct
  source 1..17: mol_type = other RNA
ccctgacccgtctaata

SEQ ID NO: 2 - RNA, 21 nt, synthetic construct
  source 1..21: mol_type = other RNA
gaaggaagtcatcgtgcccaa